(19) United States
(12) Patent Application Publication
(10) Pub. No.: US 2023/0184743 A1
(43) Pub. Date: Jun. 15, 2023

(54) SCREENING METHODS TO IDENTIFY SMALL MOLECULE COMPOUNDS THAT PROMOTE OR INHIBIT THE GROWTH OF CIRCULATING TUMOR CELLS, AND USES THEREOF

(71) Applicant: University of Southern California, Los Angeles, CA (US)

(72) Inventors: Min Yu, Los Angeles, CA (US); Teng Teng, Los Angeles, CA (US); Ebony Flowers, Los Angeles, CA (US)

(21) Appl. No.: 17/912,313

(22) PCT Filed: Mar. 16, 2021
(86) PCT No.: PCT/US21/22613

§ 371 Date: Sep. 16, 2022

Related U.S. Application Data
(60) Provisional application No. 62/990,445, filed on Mar. 16, 2020.

(51) Int. Cl. G01N 33/50; C12N 5/09; A61P 35/00; A61K 31/519

(52) U.S. Cl. CPC G01N 33/5011; G01N 33/5044; C12N 5/0693; A61P 35/00; A61K 31/519; G01N 2500/10

(57) ABSTRACT

The disclosure provides screening methods to identify small molecule compounds that can promote single circulating tumor cells (CTCs) proliferation, or alternatively inhibit proliferation by CTCs, and uses thereof, including as treatment options for cancer.

### CROSS REFERENCE TO RELATED APPLICATIONS

This application claims priority under 35 U.S.C. § 119 from Provisional Application Ser. No. 62/990,445, filed Mar. 16, 2020, the disclosures of which are incorporated herein by reference.

### TECHNICAL FIELD

The disclosure provides screening methods to identify small molecule compounds that can promote single circulating tumor cells (CTCs) proliferation, or alternatively inhibit proliferation by CTCs, and uses thereof, including as treatment options for cancer.

### BACKGROUND

Circulating tumor cells (CTCs) can be isolated via a minimally invasive blood draw and are considered a “liquid biopsy” of their originating solid tumors. CTCs contain a small subset of metastatic precursors that can form metastases in secondary organs, and provide a resource to identify mechanisms underlying metastasis-initiating properties. Despite technological advancements that allow for highly sensitive approaches of detection and isolation, CTCs are very rare and often present as single cells, posing an extreme challenge for ex vivo expansion after isolation.

### SUMMARY

The disclosure provides screening methods to identify small molecule compounds that can promote single circulating tumor cells (CTCs) proliferation, or alternatively inhibit proliferation by CTCs. It was further found herein, that N-acetylcysteine (NAC) and other antioxidants identified using the screening methods of the disclosure can promote ex vivo expansion of single CTCs, facilitating subsequent functional analyses. It was also found herein, that 3-(3-(2-(3,4,5-Trimethoxy-phenylamino)-7H-pyrrolo[2,3-d]pyrimidin-7-yl)phenyl)propionitrile identified using the screening methods of the disclosure can inhibit proliferation and decrease survivability of CTCs.

In a particular embodiment, the disclosure provides a method to screen small molecules for their effect on the proliferation of single circulating tumor cells (CTCs), comprising: isolating single CTCs from a CTC-based cell line using fluorescence activated cell sorting (FACS); culturing isolated single CTCs with a small molecule compound or without; evaluating CTC cell proliferation at one or more time points with the small molecule compound or without; wherein an increase in CTC cell proliferation with a small molecule compound in comparison to CTC cell proliferation in media not containing a compound indicates that the small molecule compound promotes proliferation of CTCs; and wherein a decrease in CTC cell proliferation with a small molecule compound in comparison to CTC cell proliferation in media not containing a compound indicates that the small molecule compound inhibits proliferation of CTCs. In another embodiment, the single CTCs are isolated from a CTC-based cell line. In yet another embodiment, the CTC-based cell line is selected from BRx50, BRx68, BRx07, BRx42 and BRx142. In a further embodiment, the single CTCs are fluorescently labeled using 7-AAD prior to FACS. In yet a further embodiment, single CTCs are cultured with a small molecule compound having a concentration from 0.1 mM to 5 mM. In another embodiment, the media used to culture the single CTCs with the small molecule compound is changed every three days with fresh compound. In yet another embodiment, the CTC cell proliferation is evaluated every 6 days. In another embodiment, the CTC cell proliferation is evaluated from 24 days.

In a certain embodiment, the disclosure also provides a method to increase proliferation of circulating tumor cells (CTCs) comprising: culturing CTCs in a medium comprising N-Acetyl-L-Cysteine (NAC), P1C2, and/or diclofenac sodium. In yet a further embodiment, the medium comprises 300 μM of NAC.

In a particular embodiment, the disclosure further provides a method to inhibit the proliferation and/or decrease the survivability of circulating tumor cells (CTCs) comprising: contacting the CTCs with a therapeutically effective amount of 3-(3-(2-(3,4,5-Trimethoxy-phenylamino)-7H-pyrrolo[2,3-d]pyrimidin-7-yl)phenyl)propionitrile. In another embodiment, the method is carried out in vitro, ex vivo or in vivo. In yet another embodiment, a pharmaceutical composition comprises the therapeutically effective amount of 3-(3-(2-(3,4,5-Trimethoxy-phenylamino)-7H-pyrrolo[2,3-d]pyrimidin-7-yl)phenyl)propionitrile. In another embodiment, the pharmaceutical composition comprising therapeutically effective amount of 3-(3-(2-(3,4,5-Trimethoxy-phenylamino)-7H-pyrrolo[2,3-d]pyrimidin-7-yl)phenyl)propionitrile is administered in vivo to a subject in need thereof. In yet another embodiment, the subject has advanced metastatic cancer.

### DESCRIPTION OF DRAWINGS

FIG. 1A-C provides an illustration of the single cell drug screen process. (A) Illustration of the small molecule screening process (top panel) and the summary of results from the first and second round screenings (bottom panel). (B) Representative phase contrast images of the growth of a single BRx68 CTC at different time points. Scale bar: 200 μm. (C) Representative phase contrast and GFP-fluorescent images of CTC clones generated from different CTC lines (BRx50, BRx68, BRx07, and BRx42). BRx42 cells used are not GFP transduced.

FIG. 2A-B demonstrates optimization of NAC concentration. (A) Graph showing AUC measurement of the proliferation of single BRx68 cells over 24 days with various NAC concentrations. *P<0.05. (B) Graph showing AUC measurement of the proliferation of single BRx68 cells over 24 days with various NAC concentrations in a separate batch. *P<0.05.

FIG. 3A-D shows that NAC and NAC+P1C2 combined promote growth of single CTCs from multiple lines. Graph showing AUC measurement of the proliferation of single CTCs from 4 different CTC lines over 24 days with NAC 300 μM (A), NAC+P1C2 (B), P1G3 (C), or P4D8 (D). * P<0.05; ** P<0.01; *** P<0.001. P values were obtained by a Kruskal-Wallis test adjusted by Benjamini-Hochberg Procedure for multiple testing.

FIG. 4A-D shows that NAC and NAC+P1C2 combined promote growth of small numbers of CTCs isolated from blood samples. BRx42 (A, B) or BRx50 (C, D) spiked into healthy volunteers' blood were isolated and cultured in either CTC media (control) or CTC media containing either NAC or NAC+P1C2 for 20 days. Representative images of BRx42 (A) or BRx50 (C) in 96 well plates at day 20. Graphs showing CTC growth ratio in each well of BRx42 (B) or BRx50 (D) at day 20. Each well contains 1-11 CTCs at day 0. Arrows point to single CTCs in the BRx42 CT condition. CT: N=30, P1C2: N=26, NAC: N=26 Mixture (P1C2+NAC): N=27. Scale bar: 200 μm; mean±s.e.m. P values were obtained with two-tailed unpaired t-test, F of F test both >0.05; ns, non-significant; * P<0.05; ** P<0.01; *** P<0.001; **** P<0.0001.

FIG. 5A-C demonstrates pretreatment with short time NAC dose not change the tumorigenicity of CTCs. (A) Total glutathione in cells treated with NAC for either 6 or 13 days relative to control untreated cells. (B) The graph shows the tumor growth kinetics of single BRx68 clones generated with (NAC) or without (control) NAC after the first 24 days (NAC group: N=3, control group N=5). P=0.7868. P value was analyzed by two-way ANOVA with RM by columns between 2 groups at matched time point. Interaction between groups has been tested. (C) Representative images of Hematoxylin & Eosin staining of the primary tumor generated in control and NAC groups. Scale bar: 100 μm.

FIG. 6A-E provides RNA-seq analysis of pools of clones at days 6 and 13. (A) PCA plot of RNA-seq results from pools of single cell clones at days 6 and 13 in control and molecule treated conditions, including 2 antioxidants (NAC and P4D8) and 2 COX inhibitors (P1C2 and P1G7). (B) Graphs of IPA analysis of enriched molecular and cellular functions of differentially expressed genes between days 13 and 6. (C) Graphs of IPA analysis of enriched molecular and cellular functions of differentially expressed genes between molecule treated conditions and control at day 6. (D) Heatmap for all samples in B based on previously published quiescent and senescent gene signature. Clustering is based on the Euclidean distance between samples. (E) Heatmap for NAC-treated cells at day 13 based on previously published senescent gene signature. Rows represent Z-score of normalized expression values of the marker genes.

FIG. 7 provides a graph showing AUC measurement of the inhibition of the proliferation of single CTCs from 4 different CTC lines over 24 days with Casein Kinase II Inhibitor IV 3-(3-(2-(3,4,5-Trimethoxy-phenylamino)-7H-pyrrolo[2,3-d]pyrimidin-7-yl)phenyl)propionitrile.

FIG. 8A-B shows NAC treatment rescued a CTC clone isolated from a patient with breast cancer. (A) Two single CTCs were isolated from a tube of blood from a patient with breast cancer were cultured in separate wells. Phase-contrast microscope images and Cell tracker green channel for single-cell clone 1 (left) and 2 (right) at day 7 and day 14 in culture under regular media. Phase-contrast image for single-cell clone 2 at day 60 was shown (after 8 weeks of NAC treatment). (B) Heatmap showing CNV profiles of 8 single cells isolated from single CTC clone 2 after 12 weeks of NAC treatment.

FIG. 9A-C shows the effects of a compound of Formula II (p1B5) on CTC cells at 1 μM. (A) Caspase-3 activity. (B) Ki-67 activity. (C) p21 activity.

FIG. 10A-C shows β-galactosidase activity in various cell types when cultured with p1B5 at various concentrations or CX-4945. (A) MCF7 cells. (B) MDA-MB-231 cells. (C) T47D cells.

FIG. 11A-B shows a kill curve for various cell lines at (A) 3 days and (B) 5 days of culture.

### DETAILED DESCRIPTION

As used herein and in the appended claims, the singular forms “a,” “an,” and “the” include plural referents unless the context clearly dictates otherwise. Thus, for example, reference to “an inhibitor” includes a plurality of such inhibitors and reference to “the small molecule” includes reference to one or more small molecules and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

It should be understood that this disclosure is not limited to the particular methodology, protocols, and reagents, etc., described herein and as such may vary. The terminology used herein is for the purpose of describing particular embodiments only and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with percentages means ±1%.

For purposes of the disclosure the term “cancer” will be used to encompass cell proliferative disorders, neoplasms, precancerous cell disorders and cancers, unless specifically delineated otherwise. Thus, a “cancer” refers to any cell that undergoes aberrant cell proliferation that can lead to metastasis or tumor growth. Exemplary cancers include but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodermal tumors, visual pathway and hypothalamic glioma, breast cancer, including triple negative breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, soft tissue sarcoma, uterine cancer, uterine sarcoma, skin cancer (non-melanoma), skin cancer (melanoma), papillomas, actinic keratosis and keratoacanthomas, merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor. In a particular embodiment, the cancer is advanced metastatic cancer.

Circulating tumor cells (CTCs) are cancer cells shed from primary or metastatic lesions into systemic circulation. Since CTCs can be shed from multiple active tumor lesions, and they contain precursors that can eventually initiate metastasis, CTCs are considered a liquid biopsy for solid tumors. It has been shown that high numbers of CTCs correlate with a worse prognosis in several types of cancer. Despite significant variability between patients and disease stages, CTCs are generally very rare. Most patients with metastatic cancers, including prostate, ovarian, breast, gastric, colorectal, bladder, renal, non-small cell lung, and pancreatic cancers, have low numbers of CTCs in a tube of blood, according to an analysis using the Cell Search platform, which captures CTCs based on EpCAM expression. Although technologies that do not solely rely on EpCAM-surface expression have been reported to capture a higher number of CTCs the quantity remains too low for downstream functional analysis in many cases.

Several studies have shown successful ex vivo expansion of CTCs isolated from patients with breast, colorectal, and prostate cancer. These CTC lines have provided sufficient amounts of material for many analyses, including xenograft analysis and drug susceptibility assessment. However, the efficiency of establishing the ex vivo culture of CTCs is extremely low, limiting its broad application to the majority of the cancer patients. This low efficiency may be due to limited quantities, low capture efficiency, the harshness of the procedure, and the vulnerability of CTCs in circulation. It has been shown that CTCs experience significant stress from high reactive oxygen species (ROS) levels, induced by the detachment of the extracellular matrix (ECM) or cell-cell connections. Cells resilient to ROS may have a higher chance of initiating metastasis. Changes in both glucose and glutamine metabolism have been found to regulate proper redox balance in CTCs and promote anchorage independent growth.

Antioxidants promote CTC survival and metastasis in lung, melanoma, and prostate cancers. In addition, CTC clusters have a greater tendency to survive and metastasize, due to cell-cell interactions. However, CTC clusters are only detected in a small percentage of patients, while single CTCs are far more common. Improved culture conditions for expanding single CTCs may help resolve their molecular and phenotypic properties.

Due to the rarity of CTCs, optimizing culture conditions for single cell expansion has been challenging. Several CTC lines have been established. These CTC lines can be maintained long-term in culture. However, when dissociated and plated as single cells, it is extremely difficult for majority of these cells to expand successfully. These CTC lines exhibit metastatic potential that represents the major metastatic lesions in corresponding patients. However, like primary tumors, CTCs are heterogeneous with different EMT and stem cell status. Hence, single CTCs were used as a platform to screen a small molecule library in order to identify compounds that promote their expansion in culture.

Provided herein, are methods to screen a library of small molecule therapeutics using single CTCs from isolated sample or from established breast cancer CTC lines in order to identify compounds that can promote or inhibit CTC expansion in vitro. Using the screening methods of the disclosure, NAC, at about 300 μM (e.g., 250-350 μM) concentration, was identified as useful for promoting single CTC growth from multiple patient-derived CTC lines. The use of NAC provides for the generation of a robust screening system for CTC analyses.

It is currently not feasible to isolate CTCs from patients sufficient to screen chemotherapeutics or other agents due to CTC rarity. Thus, large-scale screening studies cannot be performed from freshly isolated CTCs. Accordingly, the methods of the disclosure can be used to address an important problem in expanding single CTCs ex vivo by identifying agents that promote CTC expansion.

During single CTC expansion, RNA-seq analysis presented herein indicated significant metabolic changes. In small pools of growing clones of CTCs, there is an increasing transcriptomic heterogeneity, which can be reduced by molecules such as antioxidants or COX inhibitors. Antioxidants, such as NAC induced changes in metabolism, including lipid metabolism that may promote CTC proliferation.

In the studies presented herein, it was found that the constraints on proliferation are significantly reduced once single CTCs achieve a critical mass. This suggests that cell-cell contact mitigates stress. This also indicates that compounds promoting CTC proliferation can be applied in the short term, and need not confound the downstream characterization of CTC biology. The xenograft assays presented herein, further confirmed this result, in that tumorigenicity between several single cell clones from NAC and control groups were similar. The cell-type dependent effects of some compounds reflect the inter-patient heterogeneity of CTCs. While some compounds promote cell proliferation only in certain CTC lines, other compounds have either promoting or inhibitory effects, depending on the patient cell line. It was further shown that inter-patient heterogeneity in driver mutations in CTCs and associated patient-dependent drug susceptibilities. While reducing oxidative stress is a common need for CTCs, other specific pathways are quite distinct among patients.

Using the screening methods of the disclosure, NAC and other compounds with antioxidant properties were found to promote single CTC proliferation, likely by altering cell metabolism to facilitate survival and growth. These compounds are useful in expanding CTCs from a broader cancer patient cohort, thereby advancing the understanding of the biological properties of these rare and clinically important cells.

The disclosure provides methods of isolating, culturing and/or screening CTC cells. The method includes obtaining a biological sample from a subject. In one embodiment, the subject has been diagnosed with a cancer. In another embodiment, the subject is suspected of having cancer. In still another embodiment, the sample is a blood sample. The biological sample, is processed either via a positive selection process or negative selection process, depending upon the cancer cell type of interest to identify and isolate CTCs. Method of isolating CTCs can utilize commercially available kits, antibodies (e.g., anti-CD56), stains and the like. Once isolated CTCs are cultured in about 300 μM (e.g., 200-400 μM or any value there between) NAC in RPMI 1640 medium, supplemented with EGF (20 ng/mL), bFGF (20 ng/mL), 1× B27 and 1× antibiotic/antimycotic, in 4% 02 and 5% CO₂ for about 24 days (e.g., 18-30 days) after which the cells were switched to media lacking NAC. In screening assays, following isolating and culturing, a test agent is added to the CTC culture and the affect the test agent has on the culture is determined compared to a similar culture lacking the test agent. Readouts of the test agent can include cell growth, apoptosis, migration, infiltration, proliferation etc. In some instances, gene expression readouts can be examined to see effects a test compound has on genome expression. In some embodiments, the test agent is an anti-cancer agent including a chemotherapeutic agent, an anti-cancer biological agent (e.g., siRNA, antibodies, non-immunoglobulin binding domains etc.). In one embodiment, the assay method is used to determine susceptibility to potentially metastatic CTC in a patient sample for a proposed therapy (e.g., chemotherapy). In this embodiment, the subject's blood is obtained, CTCs isolated and culture in NAC to expand the isolated CTCs, NAC is subsequently removed and then the CTC are contacted with a panel of potential anti-cancer agents. Once a suitable anti-cancer agent is identified, the subject is then treated with that anti-cancer agent.

Using the methods and compositions of the disclosure a compound was identified that inhibit CTC growth and proliferation and induced senescence. In one embodiment, the disclosure provides a compound having the general structure of Formula I:

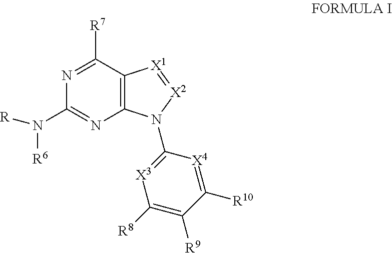

wherein R is an unsubstituted or substituted heterocycle, unsubstituted or substituted aryl, unsubstituted or substituted cycloalkyl, or unsubstituted or substituted cycloalkenyl, X¹ is N or CR¹¹, X² is N or CR¹², X³ is N or CR¹³, X⁴ is N or CR¹⁴; R⁶-R⁷ are each independently H, optionally substituted (C₁-C₆)alkyl, an optionally substituted (C₁-C₆) hetero-alkyl, an optionally substituted (C₁-C₆)alkenyl, an optionally substituted (C₁-C₆) hetero-alkenyl, an optionally substituted (C₁-C₆)alkynyl, an optionally substituted (C₁-C₆) hetero-alkynyl, an optionally substituted aryl, an optionally substituted (C₃-C₆)cycloalkyl, an optionally substituted (C₃-C₆)cycloalkenyl, and an optionally substituted heterocycle; R⁸-R¹⁰ are each independently H, optionally substituted (C₁-C₆)alkyl, —(CH₂)_{y}-nitrile; R¹¹-R¹⁴ are each independently H, optionally substituted (C₁-C₆) alkyl, an optionally substituted (C₁-C₆) hetero-alkyl, an optionally substituted (C₁-C₆) alkenyl, an optionally substituted (C₁-C₆) hetero-alkenyl, an optionally substituted (C₁-C₆)alkynyl, an optionally substituted (C₁-C₆) hetero-alkynyl, an optionally substituted aryl, an optionally substituted (C₃-C₆)cycloalkyl, an optionally substituted (C₃-C₆) cycloalkenyl, and an optionally substituted heterocycle; and y is an integer selected from 0, 1, 2, 3, 4, 5, and 6.

In another embodiment, the disclosure provides a compound of Formula I(a):

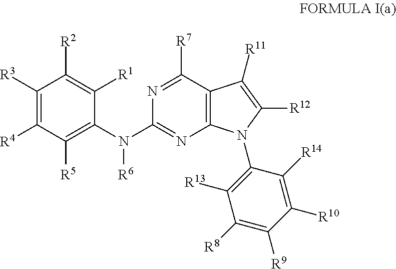

wherein R¹-R⁵ are each independently selected from H, a (C₁-C₆)alkoxy, optionally substituted (C₁-C₆)alkyl, an optionally substituted (C₁-C₆) hetero-alkyl, an optionally substituted (C₁-C₆)alkenyl, an optionally substituted (C₁-C₆) hetero-alkenyl, an optionally substituted (C₁-C₆)alkynyl, an optionally substituted (C₁-C₆) hetero-alkynyl, an optionally substituted aryl, an optionally substituted (C₃-C₆) cycloalkyl, an optionally substituted (C₃-C₆)cycloalkenyl, and an optionally substituted heterocycle; R⁶-R⁷ are each independently H, optionally substituted (C₁-C₆)alkyl, an optionally substituted (C₁-C₆) hetero-alkyl, an optionally substituted (C₁-C₆)alkenyl, an optionally substituted (C₁-C₆) hetero-alkenyl, an optionally substituted (C₁-C₆) alkynyl, an optionally substituted (C₁-C₆) hetero-alkynyl, an optionally substituted aryl, an optionally substituted (C₃-C₆) cycloalkyl, an optionally substituted (C₃-C₆) cycloalkenyl, and an optionally substituted heterocycle; R⁸-R¹⁰ are each independently H, optionally substituted (C₁-C₆) alkyl, —(CH₂)_{y}-nitrile; R¹¹-R¹⁴ are each independently H, optionally substituted (C₁-C₆)alkyl, an optionally substituted (C₁-C₆) hetero-alkyl, an optionally substituted (C₁-C₆) alkenyl, an optionally substituted (C₁-C₆) hetero-alkenyl, an optionally substituted (C₁-C₆)alkynyl, an optionally substituted (C₁-C₆) hetero-alkynyl, an optionally substituted aryl, an optionally substituted (C₃-C₆)cycloalkyl, an optionally substituted (C₃-C₆)cycloalkenyl, and an optionally substituted heterocycle; and y is an integer selected from 0, 1, 2, 3, 4, 5, and 6.

In another embodiment, the disclosure provides a compound of Formula II:

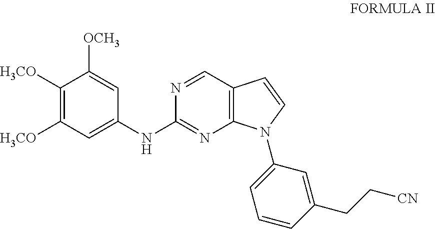 [SMILES: COc1cc(Nc2ncc3ccn(-c4cccc(CCC#N)c4)c3n2)cc(OC)c1OC]

The screening methods of the disclosure identified compounds comprising Formula I that inhibit the growth of circulating tumor cells (CTCs), e.g., 3-(3-(2-(3,4,5-Trimethoxy-phenylamino)-7H-pyrrolo[2,3-d]pyrimidin-7-yl)phenyl)propionitrile.

The disclosure provides a method of treating a subject with cancer (e.g., breast cancer, prostate cancer, lung cancer etc.) comprising administering to the subject a compound of Formula I, I(a) or II in an amount effective to induce senescence of CTC in the subject. In one embodiment, the disclosure comprises administering a pharmaceutical composition comprising a compound of Formula I, I (a) or II.

The disclosure provides a method for inhibiting CTC growth and/or survivability by contacting or administering a compound of the disclosure, comprising administering a therapeutically effective amount of a CTC inhibitory growth compound disclosed herein (a compound of Formula I; I(a); or II, 3-(3-(2-(3,4,5-Trimethoxy-phenylamino)-7H-pyrrolo[2,3-d]pyrimidin-7-yl)phenyl)propionitrile) to a subject who has cancer (e.g., advanced metastatic cancer). The term “inhibiting” means preventing or ameliorating a sign or symptoms of a cancer and/or a neoplastic disorder (e.g., tumor growth, cancer cell proliferation and/or migration, cancer cell metastasis, and the like) and/or inducing senescence of CTCs in the subject.

The disclosure also provides a method for inhibiting the growth of CTC cells by contacting the CTC cells with an inhibiting effective amount of a compound disclosed herein. The term “contacting” refers to exposing the cells (e.g., CTC cells) to an agent. Contacting can occur in vivo, for example, by administering a compound of the disclosure to a subject afflicted with cancer. In vivo contacting includes both parenteral as well as topical. “Inhibiting” or “inhibiting effective amount” refers to the amount of a compound disclosed herein that is sufficient to cause, for example, CTC cell death, inhibition of growth and/or migration and/or inhibition or prevention of metastasis.

A pharmaceutical composition comprising a compound of the disclosure can be in a form suitable for administration to a subject using carriers, excipients, and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol, and polyhydric alcohols. Intravenous vehicles include fluid and nutrient replenishers. Preservatives include antimicrobial, chelating agents, and inert gases. Other pharmaceutically acceptable carriers include aqueous solutions, non-toxic excipients, including salts, preservatives, buffers and the like, as described, for instance, in Remington's Pharmaceutical Sciences, 15th ed., Easton: Mack Publishing Co., 1405-1412, 1461-1487 (1975), and The National Formulary XIV., 14th ed., Washington: American Pharmaceutical Association (1975), the contents of which are hereby incorporated by reference. The pH and exact concentration of the various components of the pharmaceutical composition are adjusted according to routine skills in the art. See Goodman and Gilman's, The Pharmacological Basis for Therapeutics (7th ed.).

The disclosure further provides for a pharmaceutical composition comprising a compound disclosed herein that can be administered in a convenient manner, such as by injection (subcutaneous, intravenous, etc.), oral administration, inhalation, transdermal application, or rectal administration. Depending on the route of administration, the pharmaceutical composition can be coated with a material to protect the pharmaceutical composition from the action of enzymes, acids, and other natural conditions that may inactivate the pharmaceutical composition. The pharmaceutical composition can also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersions. In all cases, the composition should be sterile and should be fluid to the extent that easy syringability exists. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size, in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be typical to include isotonic agents, for example, sugars, polyalcohols, such as mannitol, sorbitol, or sodium chloride in the composition. Prolonged absorption of the injectable compositions can be brought about by including in the composition an agent that delays absorption, for example, aluminum monostearate and gelatin.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the pharmaceutical composition into a sterile vehicle that contains a basic dispersion medium and the required other ingredients from those enumerated above.

The pharmaceutical composition can be orally administered, for example, with an inert diluent or an assimilable edible carrier. The pharmaceutical composition and other ingredients can also be enclosed in a hard or soft-shell gelatin capsule, compressed into tablets, or incorporated directly into the individual's diet. For oral therapeutic administration, the pharmaceutical composition can be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations can, of course, be varied and can conveniently be between about 5% to about 80% of the weight of the unit.

The tablets, troches, pills, capsules, and the like can also contain the following: a binder, such as gum gragacanth, acacia, corn starch, or gelatin; excipients such as dicalcium phosphate; a disintegrating agent, such as corn starch, potato starch, alginic acid, and the like; a lubricant, such as magnesium stearate; and a sweetening agent, such as sucrose, lactose or saccharin, or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar, or both. A syrup or elixir can contain the agent, sucrose as a sweetening agent, methyl and propylparabens as preservatives, a dye, and flavoring, such as cherry or orange flavor. Of course, any material used in preparing any dosage unit form should be pharmaceutically pure and substantially non-toxic/biocompatible in the amounts employed. In addition, the pharmaceutical composition can be incorporated into sustained-release preparations and formulations.

Thus, a “pharmaceutically acceptable carrier” is intended to include solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the pharmaceutical composition, use thereof in the therapeutic compositions and methods of treatment is contemplated. Supplementary active compounds can also be incorporated into the compositions.

It is especially advantageous to formulate parenteral compositions in dosage unit form for ease of administration and uniformity of dosage. “Dosage unit form” as used herein, refers to physically discrete units suited as unitary dosages for the individual to be treated; each unit containing a predetermined quantity of pharmaceutical composition is calculated to produce the desired therapeutic effect in association with the required pharmaceutical carrier. The specification for the dosage unit forms of the disclosure are related to the characteristics of the pharmaceutical composition and the particular therapeutic effect to be achieve.

The principal pharmaceutical composition is compounded for convenient and effective administration in effective amounts with a suitable pharmaceutically acceptable carrier in an acceptable dosage unit. In the case of compositions containing supplementary active ingredients, the dosages are determined by reference to the usual dose and manner of administration of the said ingredients.

For use in biological applications described herein for modulating CDC growth, kits and articles of manufacture are also described herein. Such kits can comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers can be formed from a variety of materials such as glass or plastic.

For example, the container(s) can comprise one or more CDC growth modulating agents described herein, optionally in a composition or in combination with another agent as disclosed herein, e.g., single stranded DNA guide strands. The container(s) optionally have a sterile access port (for example the container can be an intravenous solution bag or a vial having a stopper pierceable by a hypodermic injection needle). Such kits optionally comprise a RecAx complex disclosed herein with an identifying description or label or instructions relating to its use in the methods described herein.

A kit will typically comprise one or more additional containers, each with one or more of various materials (such as reagents, optionally in concentrated form, and/or devices) desirable from a commercial and user standpoint for use of a compound described herein. Non-limiting examples of such materials include, but are not limited to, buffers, diluents, filters, needles, syringes; carrier, package, container, vial and/or tube labels listing contents and/or instructions for use, and package inserts with instructions for use. A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific application. The label can also indicate directions for use of the contents, such as in the methods described herein.

The following examples are intended to illustrate but not limit the disclosure. While they are typical of those that might be used, other procedures known to those skilled in the art may alternatively be used.

### EXAMPLES

Cell culture. CTC lines were previously derived from metastatic breast cancer patients. CTC lines were cultured in ultra-low attachment plates with RPMI 1640 medium, supplemented with EGF (20 ng/mL), bFGF (20 ng/mL), 1×B27 and 1× antibiotic/antimycotic, in 4% 02 and 5% CO₂. Single CTCs were cultured in GravityTRAP™ ULA 96 well Plates (PerkinElmer). Wells on the edges of the plates were not used to avoid influence from evaporation. Media with fresh compounds were exchanged every 3 days by inserting pipet tips onto the platform of the wells to prevent accidentally aspirating suspended CTCs at the bottom. CTC numbers were counted manually under an inverted microscope every 6 days.

FACS sorting. Cells were pelleted and resuspended into single cell suspension in 1% BSA in PBS buffer with 7-AAD. Live single CTCs were sorted directly into 96-well GravityTRAP™ ULA Plates using a MoFlo cell sorter (Beckman Coulter). An inverted microscope was used to manually confirm that there was 1 CTC per well, 1 hour after sorting.

Compounds screening. Compounds were from the StemSelect library obtained from the Choi Family Therapeutic Screening Facility at the Eli and Edythe Broad CIRM Center for Regenerative Medicine and Stem Cell Research at USC. Each compound was given a code to ensure unbiased assessment and blinded to the investigators. Compound information is listed in Appendix, at Table 1 of US Provisional Application No. U.S. 62/990,445, incorporated herein by reference). In the first-round screening, compounds were used with 1 mM concentration. Stocks of compounds (10 mM concentration in DMSO) were stored in aliquots at −80° C. All compounds were thawed and refrozen for a maximum of 2 times. In the second-round validation, fresh compounds were dissolved in DMSO (Millipore sigma), and aliquots were stored at −20° C. and used only once without refreezing. Only wells started from single cells on day 0 after sorting were used in the screening experiment. Data collection was done in batches.

Spike-in experiment. Healthy volunteers' blood samples were collected. GFP-positive CTC lines (50 cells/mL) were spiked into the blood samples from healthy volunteers, and RosetteSep™ CTC Enrichment Cocktail Containing Anti-CD56 was used to enrich CTCs from spiked-in samples. Isolated CTCs were cultured in GravityTRAP™ ULA Plate 96 wells. Each patient CTC cell line was processed by 2 different researchers.

Single CTC clones. NAC clones were generated by treating single CTCs with 300 μM NAC media for 24 days before switching to regular CTC media. Control single clone lines were established in the same batch using regular CTC media.

Xenograft assay and Hematoxyline & Eosin staining. Six-week old female NSG mice (Jackson Laboratory) were anesthetized with isoflurane and 20,000 GFP/luc-positive single clone cells in 100 μL of 1:1 PBS and Corning® Matrigel® Matrix (phenol-red free) were injected into the fourth mammary fat pad. To evaluate the growth of primary tumors, mice were intraperitoneally injected with 150 μL of d-Luciferin substrate at 30 mg/mL (Sid Labs), and imaged within 15 minutes. Signals from luciferase-tagged cells were monitored at day 0 after injection and weekly by in vivo imaging using IVIS Lumina II (Perkin Elmer) for 5 weeks. Mice were sacrificed after 8 weeks, and their organs were dissected and imaged. Primary tumors and organs were collected and fixed with 10% formalin overnight and sectioned for 5 μm thickness. H&E staining was performed using Varistain Gemini ES Automated Slide Stainer in USC's Histology Laboratory (HIST). Images were taken with a 20× objective in Keyence (BZ-II Analyser, Keyence).

Patient samples. Ten milliliters of blood was collected from each of a total of 12 patients with breast cancer (stage IV). All experiments were performed following the ethical principles. Informed consents were signed by all patients and/or their legal guardian/s.

Isolation of single CTCs. Single CTCs were detected and retrieved using the negative selection protocol of PIC&RUN assay. Briefly, 7.5 mL of blood was added to each AccuCyte Separation Tube (RareCyte) and tubes were centrifuged twice in a special device to collect buffy coats in 1 mL of CTC media. Buffy coats were stained with a cocktail of immune cell markers (IM) antibodies (CD45, CD14, and CD16) and Cell-Tracker green for 30 min at 37° C. Stained buffy coats were seeded on polyhema coated cyteslides and scanned semi-automatically using RareCyte fluorescence. When a CTC was detected, the Rarecyte ceramic tip needle was operated semi-automatically to pick up the cell of interest in a draw volume of 50 nL to 0.5 μL, and deposit it in a PCR tube contains 50 μL of media. PCR tubes were pulse spun, their contents were moved to a well of a 96-well GravityTRAP ultralow plate (InSphero) and plates were incubated at 37° C., 5% CO₂ and 4% 02 for 3 weeks. Media was replaced every 3 to 4 days and cells were checked every week.

Copy number variation analyses. Single-cell genomic copy number profiling was carried out. After lysis of individual cells in 1.5 mL lysis buffer (1:1 solution of 100 mmol/L DTT [symbol] 400 mmol/L KOH) for 2 minutes at 95° C., DNA was amplified using the WGA4 Genomeplex Single Cell Whole Genome Amplification Kit (Sigma-Aldrich, cat #. WGA4) for 23 cycles. Amplified DNA was purified using a QIAquick PCR Purification Kit (Thermo Fisher Scientific, cat #. K210012). DNA was eluted in 60 mL of TE-buffer and quantified by Qubit. Indexed Illumina sequencing libraries were constructed and barcoded using the NEBNext Ultra DNA Library Preparation Kit for Illumina (New England Biolabs, cat #. E7370L). Amplified DNA fragments with target size were selected using Agencourt AMPure XP beads (Beckman Coulter, cat #. A63880) and further confirmed by Bioanalyzer High Sensitivity DNA Analysis (Agilent, cat #5067-4626). Libraries were sequenced using the Illumina NextSeq 500. 30 bp were trimmed off the 5′ end of each read to remove the WGA4 adapter sequence before alignment to the hg19 reference genome using the Bowtie algorithm. The resulting BAM file was sorted and PCR duplicates were removed using SAMtools. Copy number profiles were obtained by sorting reads into 5,000 informatically derived “bins” across the genome with sizes normalized to contain equal lengths of uniquely mapping sequence (approximately 0.5 Mbp) according to the reference genome Hp37 (Navin et al., Nature, 472:90-4, 2011). Finally, an R script utilizing the Bioconductor package, DNAcopy_1.26.0 ([http:/]/bioconductor.org/packages/DNAcopy/), was used to normalize and segment the bin counts across each chromosome generating a genome-wide CAN profile with a resolution of approximately 1.5 Mbp (Navin et al., supra).

RNA-seq analysis. BRx68-GFP+ cells were sorted, with 1 cell per well, using the MoFlo cell sorter (Beckman Coulter). Single cells were cultured in the presence of CTC media containing either 1 μM of P1C2, P4D8, or P1G7, or 0.3 mM of NAC or a combination of 1 μM of P1C2 and 0.3 mM of NAC. Cells cultured in media containing DMSO served as a control. All cells were cultured for either 6 days or 13 days at 37° C., 5% CO₂ and 4% O₂, and media was changed every 3 days. For both day 6 and day 13 groups, only clones with more than 3 cells at day 6 were harvested and pooled to a maximum of 50 cells. Pooled samples were processed using SMARTer chemistry (SMART Seq® v4 Ultra® Low Input RNA Kit for Sequencing, Takara Clontech), according to manufacturer's instructions to generate cDNA libraries for mRNA sequencing. All cDNA samples were run on a TapeStation system (High Sensitivity D5000 DNA Analysis Kit as per manufacturer's protocol). cDNA libraries were prepared using the Nextera XT DNA Library Prep Kit (Illumina) with Nextera index kit index 1 (i7) and index 2 (i5) adapters. Libraries were sequenced on an Illumina NextSeq500 to obtain 75 bp-long single-end reads.

RNA-sequencing reads were trimmed for Nextera and Illumina adapter sequences using Trim Galore under default parameters. Trimmed reads were then mapped to the human genome build GRCh37 from Ensembl (ftp:/][/ftp.ensembl.org/pub/grch37/current/fasta/homo_sapiens/dna/Homo_sapiens.GRCh37.dna_sm.primary_as sembly.fa.gz) using STAR under optimized parameters for single-end sequenced data. Aligned reads were then counted via feature-Counts and piped into DESeq2 for normalization to sequencing depth and downstream analysis. For purposes of producing the PCA plot, count data was transformed via the vst function to eliminate the experiment-wide trend of variance over mean and the plot was produced using ggplot2. For the PCA plot, batch effects were corrected using the function removeBatchEffect from limma. For differential expression analysis, the batch effect was modeled into the design formula so as to estimate the size of the batch effect and adjust accordingly when performing differential expression, without adjusting the raw data. The contrast function was used to compare all conditions at day 13 versus day 6, each individual condition at day 13 versus day 6, or each treatment versus control at each time point. Genes with a False Discovery Rate (FDR) of 0.05 and log 2 fold change of >1.5 were piped into IPA for gene ontology analysis. DEGs with fold change ≥2 and FDR ≤0.05 identified from a previously published study are used as marker genes for quiescence and senescence states. Heatmap based on the normalized expression values of the senescent and quiescent marker genes is generated using ComplexHeatmap package. The GO enrichment analysis is performed using the Bioconductor package GOseq.

Statistical analysis. For the screening analysis, the number of cells grown over time per well per plate per batch was represented as either area under curve (AUC) or absolute number of cells. AUC was calculated for each well in each batch, by plotting days in the x-axis and the corresponding number of cells in the y-axis, then drawing a line to connect the number of cells from the starting count day to the next count day until the end day, and finally calculating the area under this line as the sum of areas of trapezoids. If there are two count days, there will be one trapezoid; if there are three count days, there will be two trapezoids, and so on. For each trapezoid, area=½*length of day interval* (number of cells in day x1+number of cells in day x2). A Wilcoxon rank sum test was performed to compare AUC of each drug to AUC of CT (control with CTC media only) or CD (control with matching amount of DMSO in CTC media) within each batch. P values were adjusted by Benjamini-Hochberg Procedure to control the FDR. In the validation experiments, drugs with adjusted P values 0.2 were considered as statistically different from CT/CD. Statistical tests were performed using R. For absolute number of cells quantification, total number of cells at end of experiment in each well in each batch was compared between treated cells and untreated control and significance was analyzed with Student t test. For other experiments, data were analyzed with Student t test, and represent the means±SEM of at least triplicate samples or averages ±SD of independent analyses, as indicated. P<0.05 was considered statistically significant. Statistical tests were performed with GraphPad Prism7 statistical software.

Glutathione measurement. A total of 1×10⁶ BRx68 cells were cultured with or without 300 μmol/L NAC for 6 or 13 days. At the end of incubation periods, cells were washed in ice cold PBS, lysed in 5% 5-sulfo-salicylic acid dehydrate by vortexing and repeating cycles of freeze-thaw, then centrifuged at 14,000 rpm for 10 minutes and supernatant was transferred to clean tubes. Glutathione (GSH) was measured using a GSH Colorimetric Detection Kit (Invitrogen) following the manufacturer's instructions.

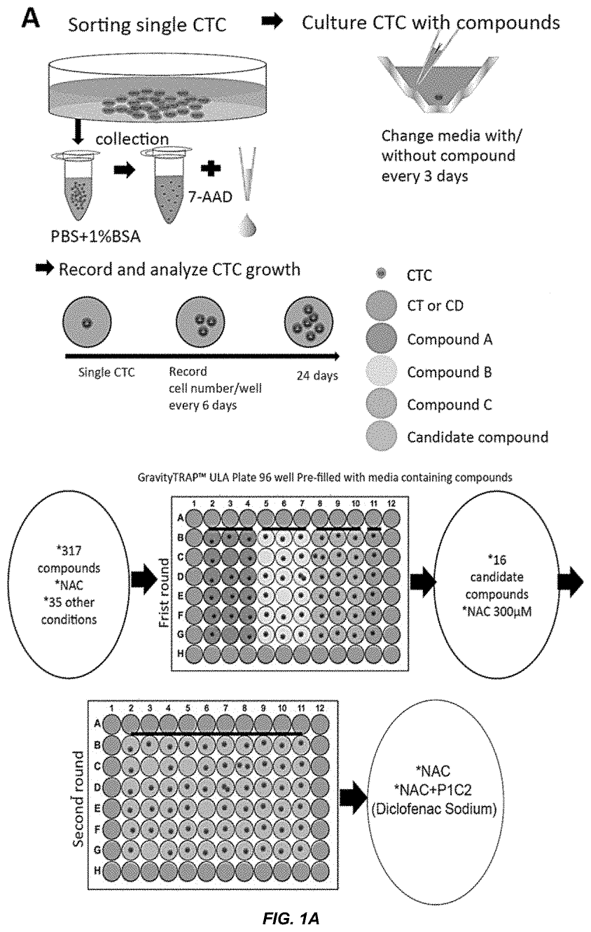

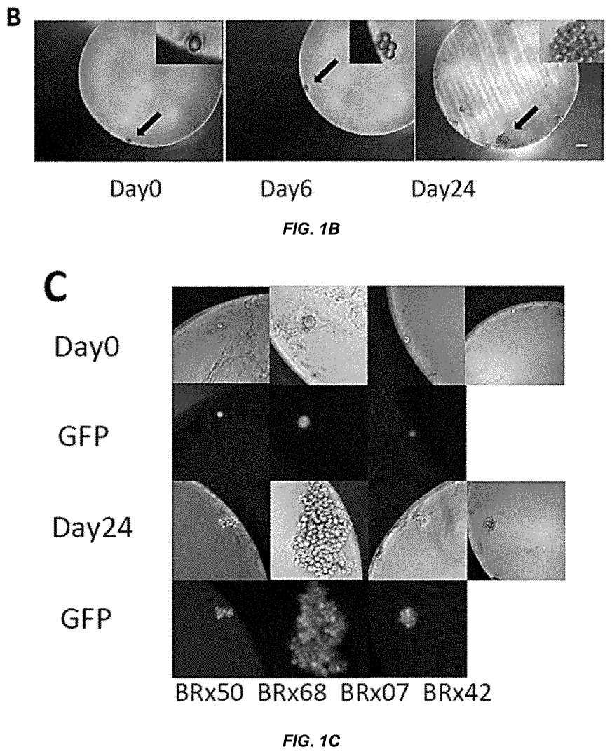

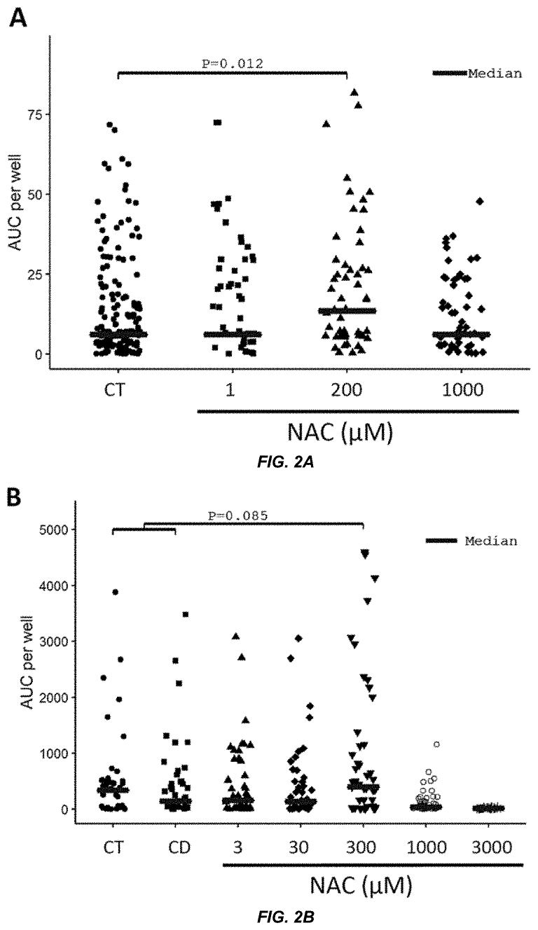

Initial low-confidence screening for single CTC expansion. A low-confidence initial screen was first performed using single CTCs sorted from our CTC lines with 317 compounds (including 15 DMSO vehicle controls with code names blinded to the experimentalist) from the StemSelect library (see Appendix, at Table 1 of US Provisional Application No. U.S. 62/990,445). Single live CTC were sorted into each well of a 96-well plate. The wells containing single cells were confirmed under a light microscope on the day after sorting and used for the screen. Cell numbers in these wells were quantified every 6 days, and media containing small molecules was replenished every 3 days (see FIG. 1A). This initial screen was performed with 12-18 wells per compound, totaling 28 successful batches with 235 plates from 3 patient-derived CTC lines (BRx68, BRx07, and BRx50). Controls were included in each batch and the compounds were tested blindly with code names. Due to significant heterogeneity in single cells, the limited number of wells tested for each compound in this initial screen is not sufficient for statistical analysis. Therefore, to identify a recurring pattern of compounds were analyzed that function in similar pathways, all the compounds that showed a higher median value of growth, based on area under the curve (AUC) calculation of cell numbers over time, compared to controls in the same batch, including CTC media only (CT) or media with vehicle DMSO (CD). Among the 130 compounds detected to have higher median AUC than the controls (see Table A (below), and Appendix, at Tables 3-4 of US Provisional Application No. U.S. 62/990,445), many of the compounds were found to have similar biological functions. Seven out of 12 cyclooxygenase (COX) inhibitors, 6 out of 11 antioxidants and free radical scavengers, and 4 out of 4 5′ adenosine monophosphate-activated protein kinase (AMPK) activators increased CTC growth in this initial screen (see Table A). Since all 3 pathways have been previously linked with reducing cellular ROS levels and in view of recent reports showing the role of antioxidants in CTC survival, a commonly used antioxidant N-acetyl-L-cysteine (NAC) was also tested. The effect of several different concentrations of NAC was first evaluated on the BRx68 line in two different batches and found the 200 μM-300 μM NAC showed the most significant effect in promoting single CTC proliferation (see FIG. 2). Therefore, NAC and several different compounds were selected from these pathways to further validate in a second-round test using a larger number of wells for statistical evaluation.

TABLE A
Compounds with similar Biological activity in the First round:

| | Number of | Number of | |
|---|---|---|---|
| | compounds with | compounds | Compounds chosen |
| Biological activity | AUC > controls | tested | for validation |
| COX inhibitor | 7 | 12 | P1G7; P1C2; P3B5; P2D9 |
| Histone deacetylase inhibitors | 8 | 15 | P1G3* |
| Sonic Hedgehog signaling antagonists/inhibitors | 7 | 10 | P2F10 |
| Antioxidants and free radical scavengers | 6 | 11 | P1C6; P1F4; P4D8 |
| Tyrosine kinase inhibtors/c-kit | 5 | 6 | P2H7 |
| SIRT Inhibitor | 5 | 7 | P1G3 |
| PARP Inhibitor | 4 | 7 | P4E9 |
| Wnt Antagonist/activates | 4 | 8 | P1B6 |
| STAT Signaling inhibitors/echancer | 4 | 7 | P1G11 |
| Ca2+ channel | 4 | 20 | |
| AMPK activator | 4 | 4 | P1A7; P2C2 |
| Proteasome-ubiquitination inhibitors | 3 | 6 | P1H5 |
| Phosphodiesterase inhibitors | 3 | 6 | |
| Phosphatase inhibitors | 3 | 9 | |
| Activates Smad & p38 | 3 | 5 | |
| Histone acetyltransferase inhibitor | 5 | 3 | P1C6 |
| Methyltransferase inhibitors | 3 | 5 | |
| UCH inhibitor | 2 | 3 | P1H5 |
| Neurogenesis inducer | 2 | 3 | |
| Adenylate cyclase inhibitors/activates | 2 | 6 | |
| CCR antagonist | 2 | 3 | |
| NF-kB activation inhibitors | 2 | 7 | |
| G-Protein antagonists | 2 | 3 | |
| Related to embryonic stem cells | 2 | 4 | |
| Related to insulin | 2 | 3 | |
| PPARα agonist/antagonists | 1 | 4 | |
| PPARα antagonists | 1 | 2 | |
| Sirtuins activates | 1 | 2 | |
| Histone acetyltransferase activate | 1 | 1 | |
| G-Protein activators/modulators | 1 | 3 | |

Validation of candidate compounds. 16 compounds plus NAC were selected from the most promising biological activity categories to validate in 4 patient-derived CTC lines (BRx07, BRx42, BRx50, and BRx68), using 36 different concentrations or combinations. This totaled 13 batches with 166 plates with 60 wells per plate.

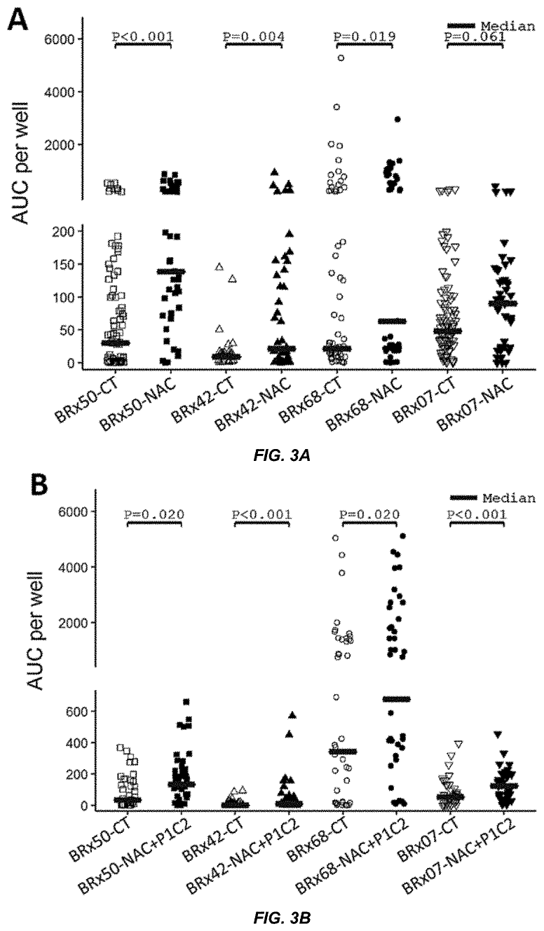

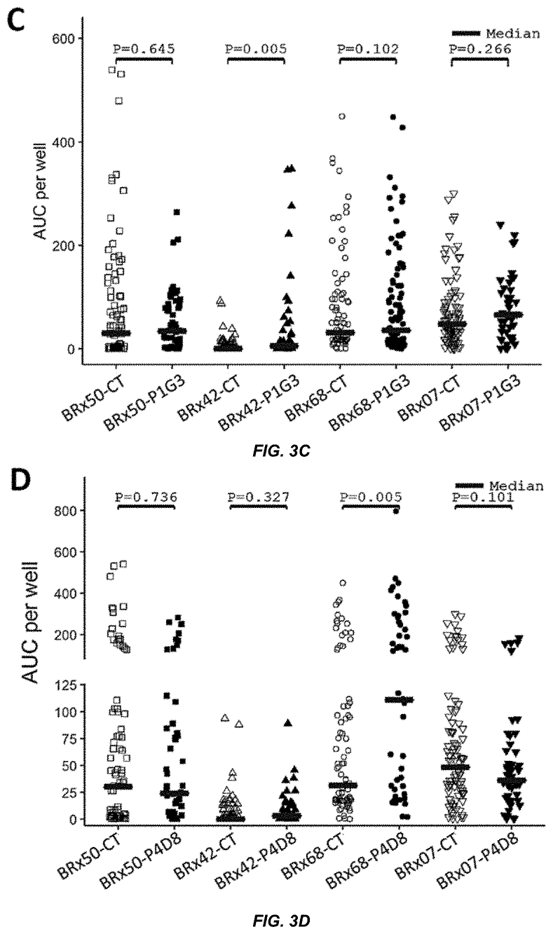

Similar to the initial screen, media were changed every 3 days, and cell numbers in each well were counted every 6 days until day 24. Results showed that the best compounds that are universal to all 4 CTC lines are NAC at 300 μM, or NAC (300 μM) in combination with the P1C2 compound-a COX-1/2 inhibitor, Diclofenac Sodium (1 μM or 0.5 μM) (see Appendix, at Tables 5 and 6 of US Provisional Application No. U.S. 62/990,445). Compared to controls, NAC or NAC+P1C2 consistently showed statistically significant improvement for single CTC expansion across many different batches for all 4 CTC lines (see FIG. 3A-B, and Appendix, Table 5 of US Provisional Application No. U.S. 62/990,445). For BRx50 and BRx42 lines, which are extremely difficult to expand as single cells, addition of these compounds can lead to the successful generation of single cell clones. Moreover, compounds were identified that increased CTC expansion in a cell line-specific manner. For example, the P1G3 compound (AGK2, a reversible inhibitor for Sirtuin-2 (SIRT2), a subclass of histone deacetylase inhibitors) can promote single cell growth specifically in BRx42 (see FIG. 3C), while the P4D8 compound (LY 231617, an antioxidant and free radical scavenger) can promote single cell growth for the BRx68 line, but inhibit growth for other lines (see FIG. 3D, and Appendix, Table 6 of US Provisional Application No. U.S. 62/990,445).

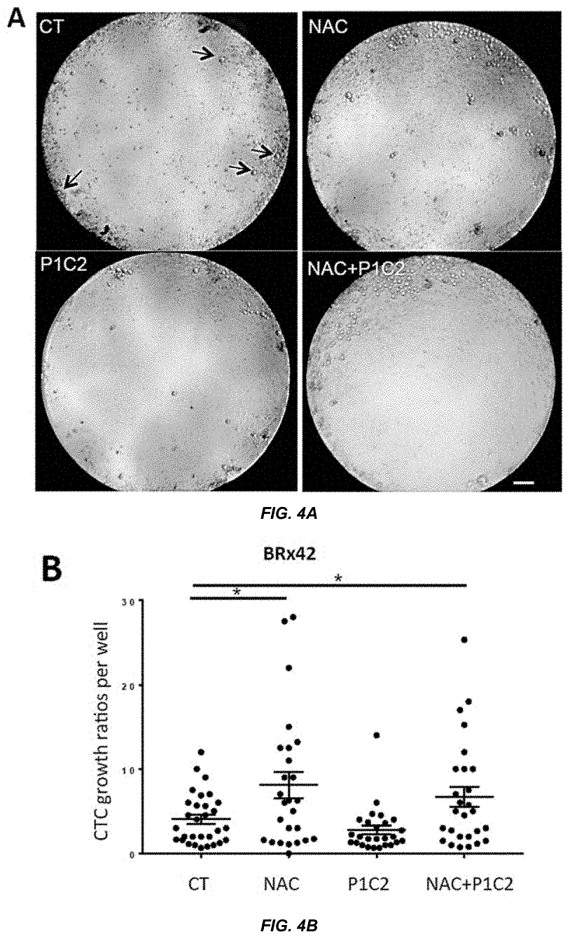

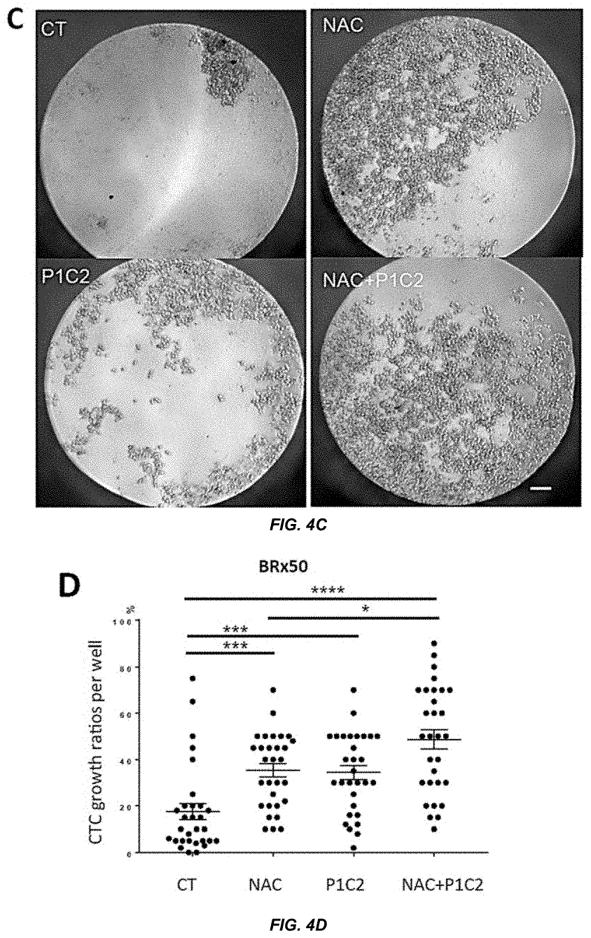

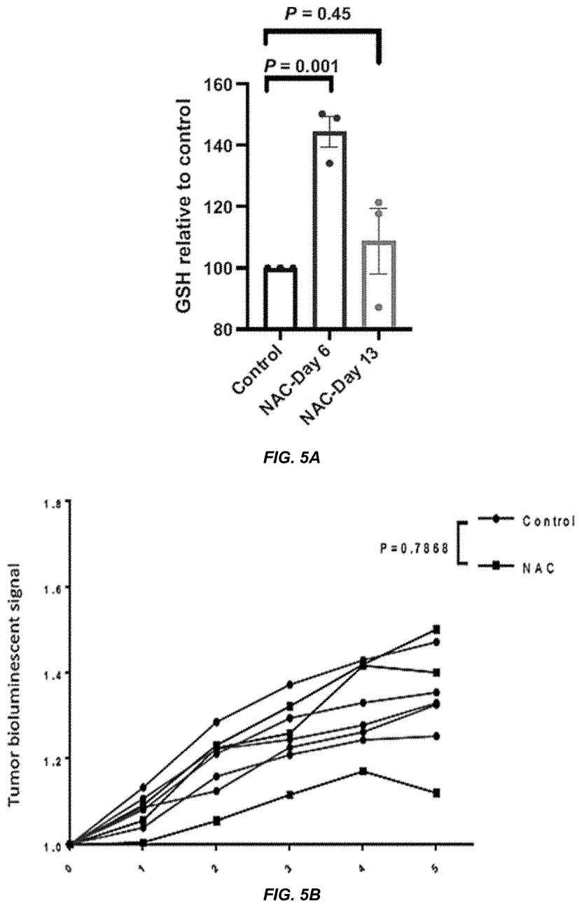

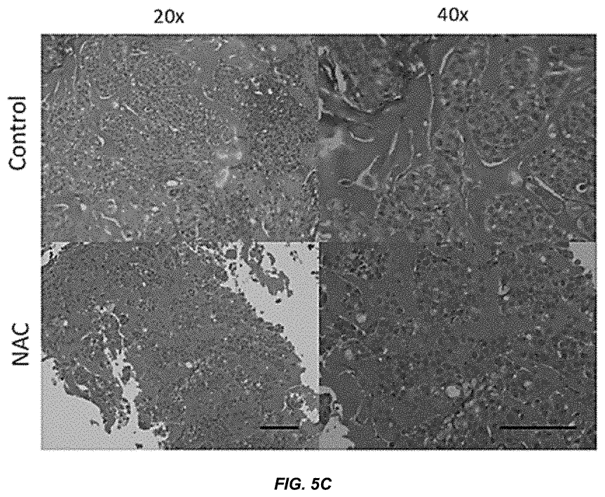

Validation on spiked-in CTCs. To mimic the CTC isolation procedure, spiked-in experiments were performed with 3 CTC lines (BRx42, BRx50 and BRx68) into healthy donors' blood. The RosettSep CTC isolation method was used to isolate the spiked CTCs, and separated isolated CTCs equally into wells with control media or with NAC or NAC plus P1C2. Compared to the control condition, NAC or NAC plus P1C2 significantly increased the growth of isolated CTCs (see FIG. 4). This confirmed the effects of these compounds on expanding CTCs that have been processed through an isolation procedure. NAC single cell clones form tumors with similar kinetics as controls. It was noticed that the most critical phase is the initial expansion from single CTCs. Once a single cell colony reaches a critical size, treatment with these compounds (NAC or NAC+P1C2) does not seem to confer additional growth advantages. Therefore, treatment of these compounds was stopped at 24 days, and then prolonged the culture to generate several single cell clones. To evaluate the tumorigenicity of the single cell clones, GFP-Luciferase tagged BRx68 single clones generated with NAC treatment or control clones were injected into the mammary fat pats of female NSG mice. The NAC treated clones generated tumors with similar growth kinetics and histology, indicating that short term treatment with NAC did not significantly affect CTCs' tumorigenicity (see FIG. 5).

NAC single-cell clones form tumors with similar kinetics as controls. The experiments shows that an important phase is the initial expansion from single CTCs. Once a single-cell colony reaches a particular size, treatment with these compounds (NAC or NAC+P1C2) does not seem to confer additional growth advantages. Indeed, total GSH level in NAC treated cells is significantly higher at day 6, but not at day 13, than that in untreated control cells (44.42±5.165, P=0.001; FIG. 5A). Therefore, after 24 days, treatment with these compounds was stopped and the cultures were prolonged to generate several single cell clones. To evaluate the tumorigenicity of the single cell clones, GFP-Luciferase-tagged BRx68 single clones, generated with NAC treatment, or control clones were injected into the mammary fat pads of female NSG mice. The NAC treated clones generated tumors with similar growth kinetics and histology, indicating that short term treatment with NAC did not significantly affect CTCs' tumorigenicity (FIGS. 5B and C).

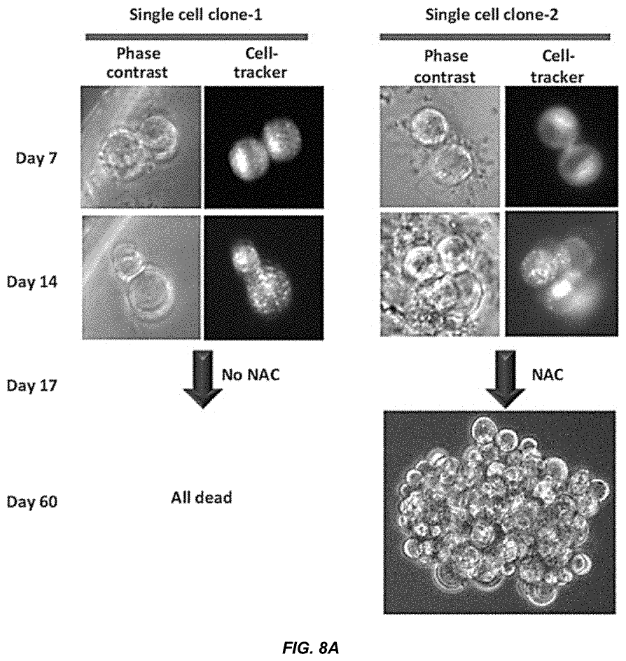

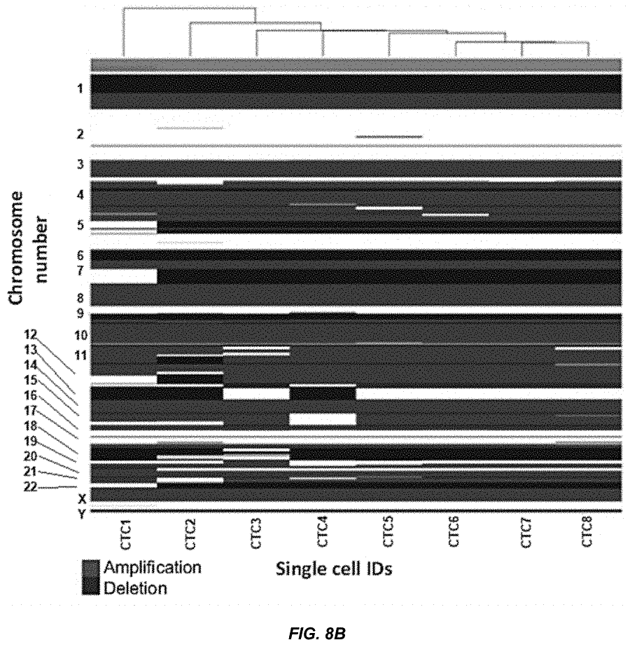

NAC rescued the proliferation of freshly isolated CTCs from a patient with breast cancer. Using the negative selection method of PIC&RUN assay live single CTCs were isolated from 12 patients with breast cancer and cultured in either NAC containing media or regular media (Table B). In one patient, two single CTCs cultured under regular media divided during the first two weeks; one cell divided once and the other divided twice (FIG. 8A). However, shortly after 2 weeks, cells started to die from both clones. In an attempt to rescue these clones, media was replaced with NAC containing media. One clone recovered and resumed proliferation for around 5 more weeks, forming a large colony of cells (FIG. 8A). To confirm that these clones are cancer cells, copy number variation (CNV) analyses was performed for 8 single cells isolated from clone 2 after 12 weeks of culture under NAC. CNV profiles of all cells showed global abnormalities resembling that of cancer cells. Moreover, the striking similarity in their global CNV patterns shows that all 8 cells were descended from a single cell (FIG. 8B).

TABLE B
Patients clinical information and number of
single CTCs cultured under NAC.

| | | | | | | Number |
|---|---|---|---|---|---|---|
| | | | | | | of |
| | | | | | | single |
| | | | | | | CTCs |
| | | | | | | cultured |
| Patient | | | Disease | Lines of | Site of | under |
| ID | Age | ER/PR/HER2 | status | treatments | metastasis | NAC |
| BC-316 | 42 | ER*/PR*/HER2* | SD^{a} | 1 | Visceral | 2 out of 12 |
| BC-318 | 29 | ER*/PR*/HER2* | PD^{b} | 5 | Visceral + Bone + Brain | 4 |
| BC-319 | 58 | ER*/PR*/HER2* | SD | 1 | Bone | 20 |
| BC-321 | 66 | ER*/PR*/HER2* | PD | 4 | Visceral + Bone | 17 |
| BC-322 | 59 | ER*/PR*/HER2* | PD | 2 | Bone | 9 |
| BC-323 | 48 | ER*/PR*/HER2* | SD | 2 | Bone | 12 |
| BC-324 | 53 | ER*/PR*/HER2* | SD | 1 | Bone + Breast | 40 |
| BC-326 | 69 | ER*/PR*/HER2* | SD | 5 | Visceral | 12 |
| BC-327 | 56 | ER*/PR* | NA^{c} | 1 | Bone | 4 |
| BC-329 | 48 | ER*/PR*/HER2* | PD | 6 | Visceral | 31 |
| BC-330 | 56 | ER*/PR*/HER2* | SD | 4 | Visceral | 57 |
| BC-331 | 62 | ER*/PR*/HER2* | SD | 3 | Visceral + Bone | 12 |

^{a}Stable disease.
^{b}Progressive disease.
^{c}Not available.

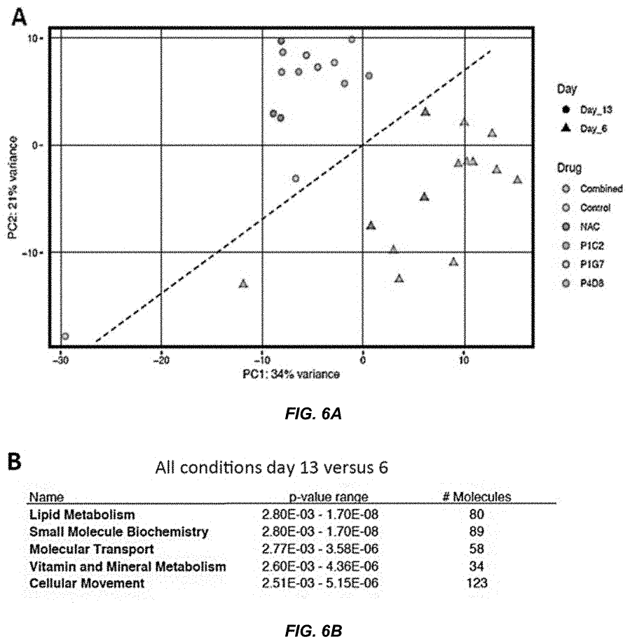

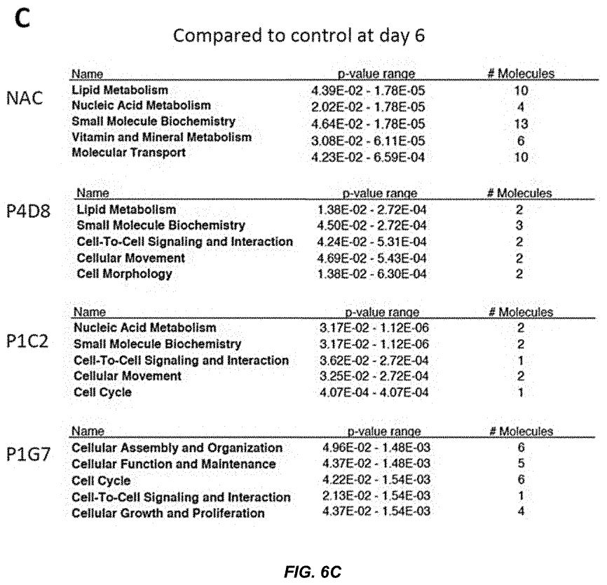

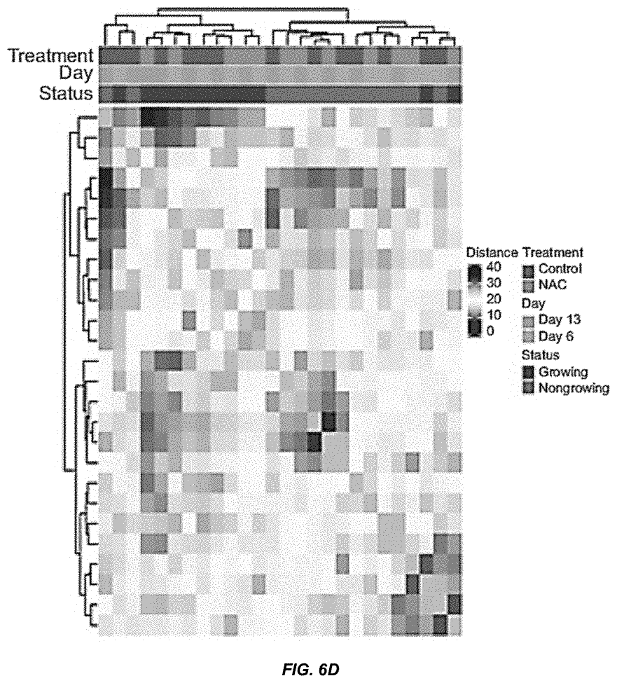

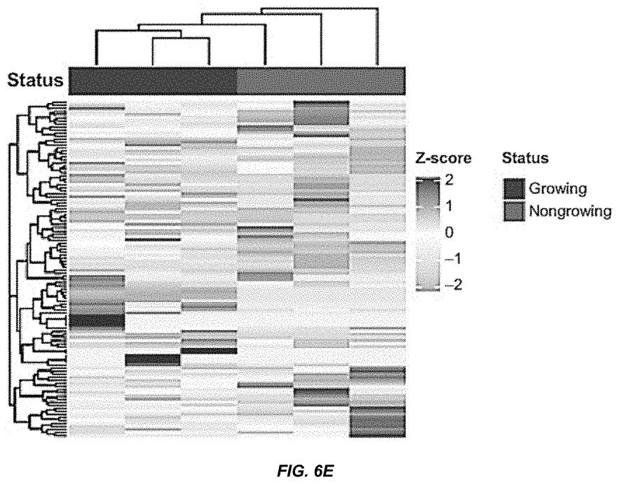

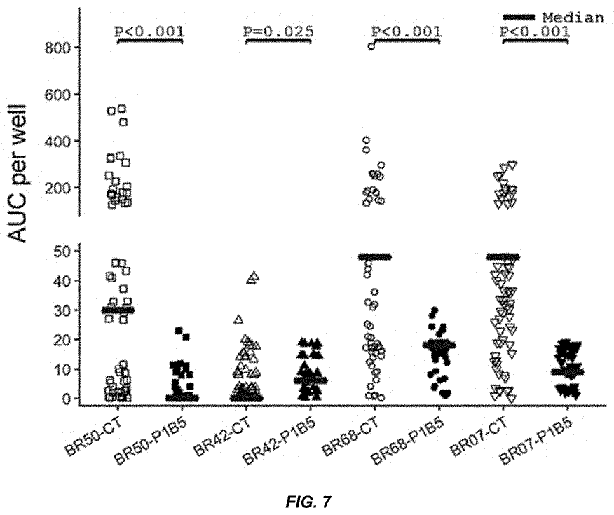

Transcriptional analysis showed metabolic changes associated with CTC expansion. To identify the transcriptional changes influenced by these molecules, RNA-seq analysis was performed of pools of growing clones from control or small molecule treated conditions. Principal component analysis (PCA) showed a clear separation of conditions at day 13 versus 6 (FIG. 6A), with a dramatic heterogeneity in the control samples at day 13. Differentially expressed genes (DEG) at day 13 versus 6 across all conditions showed enriched genes in lipid metabolism by IPA. The same lipid metabolism pathway is already enriched at day 6 in NAC and P4D8 (2 antioxidants) treatment compared with control.

Although NAC treatment significantly induced cell proliferation of single CTCs, many cells were not able to grow even under NAC treatment. This suggests that non-growing cells in both control and NAC groups may be quiescent, senescent, or a combination of both. Therefore, RNA-seq was conducted for the non-growing cells from control and NAC. As expected, PCA analysis of the growing versus non-growing cells showed a clear separation between both groups with high heterogeneity in the non-growing cells, which is expected as cell proliferation may have masked cell heterogeneity in the growing group. Heatmap clustering based on senescence and quiescence marker genes showed separation between growing and non-growing cells (FIG. 6D). As expected, the most obvious GO pathway for upregulated DEGs in growing clones at day 13 is cell-cycle regulation. By taking the control cells out of the analyses, the clustering between growing and non-growing NAC-treated clones is even more prominent with senescence markers at day 13 (FIG. 6E). Moreover, 6 upregulated DEGs in non-growing NAC-treated clones overlapped with the 45 senescence markers (P=0.079). These analyses suggest that non-growing cells contain a mixture of quiescent and senescent cells and that NAC treatment was able to push a proportion of the quiescent cells into the cell cycle, leaving behind those which are likely enriched for senescent cells.

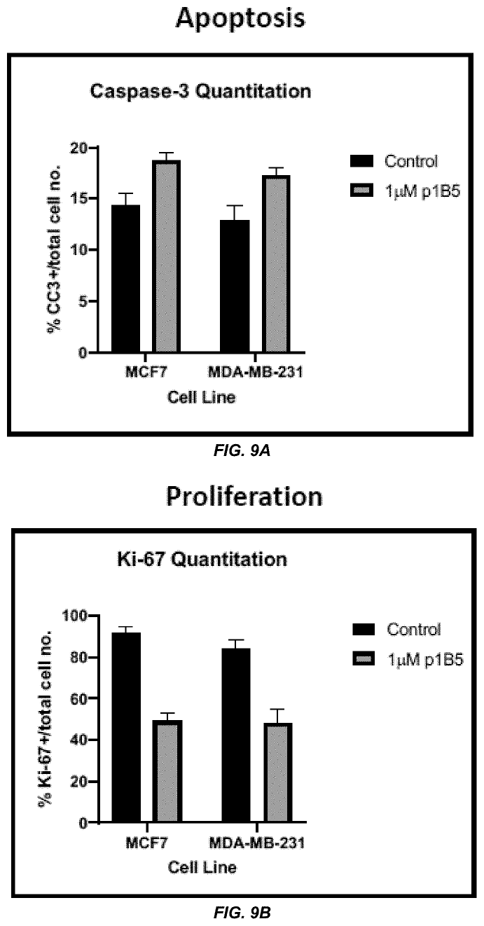

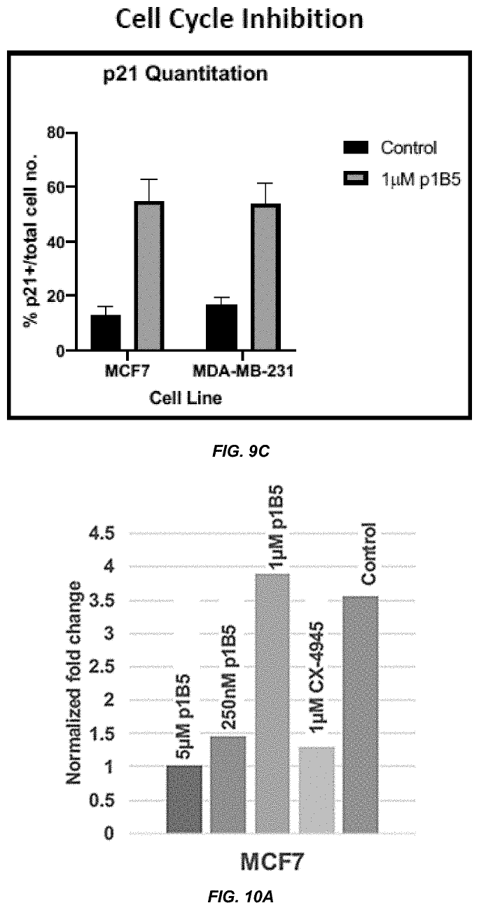

Using the assay system described herein CTC were contact with a panel of small molecule drugs and the effect of the drugs on cell growth, migration, proliferation and gene expression was measured. One compound that showed effective inhibition of CTC growth was a compound of Formula II. This was initially found in a breast cancer circulating tumor cell (CTC) growth screen. Once added at 1 uM, CTCs stopped growing and maintained at that cellular number for prolonged time. FIGS. 9A-C shows the effects of the compound of Formula II (p1B5) on MCF7 and MDA-MB-231 cells.

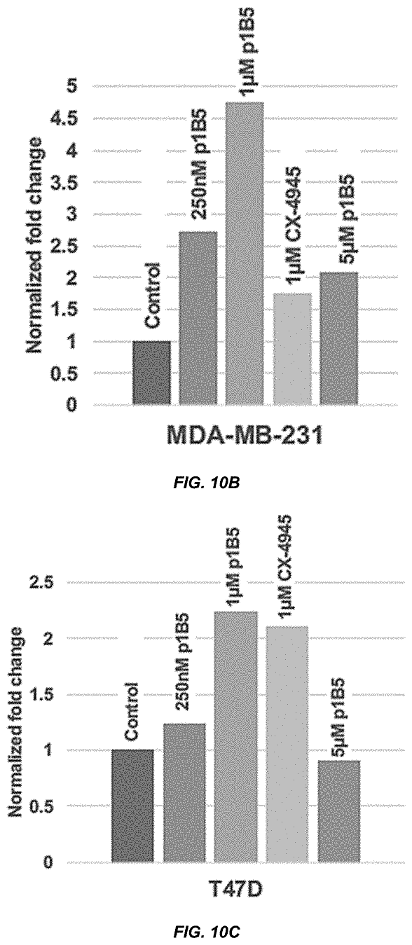

Because an arrested state in which the cell remains viable, are not stimulated to divide by serum or passage in culture, a specific cell cycle profile is illicited that differs from most damage-induced arrest processes or contact inhibition. Characteristics of senescence cells include an enlarged cell size, expression of pH-dependent β-galactosidase activity, and an altered pattern of gene expression. To determine these characteristics, CTC cells were cultured with a compound of Formula II or cx-4945 (Silmitasertib) at various concentrations and β-galactosidase activity was measured. FIGS. 10A-C shows that the compound of Formula II induced senescence by at least 3-4 fold greater than CX-4945.

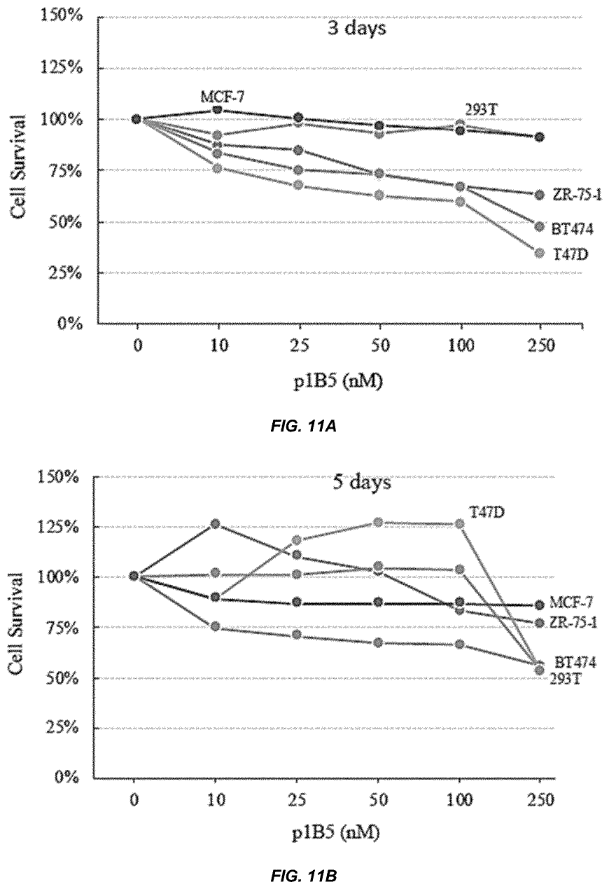

In addition, a kill curve assay was performed using 0 to 25 nM p1B5 in various cell lines (see, FIGS. 11A-B).

It will be understood that various modifications may be made without departing from the spirit and scope of this disclosure. Accordingly, other embodiments are within the scope of the following claims.

1. A method to screen small molecules for their effect on the proliferation of single circulating tumor cells (CTCs), comprising:
isolating single CTCs using fluorescence activated cell sorting (FACS);
culturing isolated single CTCs with NAC for 14-30 days and then switching the CTCs to a media lacking NAC;
culturing the CTC cells with a small molecule compound;
evaluating CTC cell proliferation at one or more time points with the small molecule compound;
wherein an increase in CTC cell proliferation with a small molecule compound in comparison to CTC cell proliferation in media lacking the small molecule compound indicates that the small molecule compound promotes proliferation of CTCs; and
wherein a decrease in CTC cell proliferation with a small molecule compound in comparison to CTC cell proliferation in media lacking the small molecule compound indicates that the small molecule compound inhibits proliferation of CTCs.

2. The method of claim 1, wherein the single CTCs are isolated from a CTC-based cell line.

3. The method of claim 2, wherein the CTC-based cell line is selected from BRx50, BRx68, BRx07, BRx42 and BRx142.

4. The method of claim 3, wherein the single CTCs are fluorescently labeled using 7-AAD prior to FACS.

5. The method of claim 1, wherein single CTCs are cultured with a small molecule compound having a concentration from 0.1 mM to 5 mM.

6. The method of claim 1, wherein the media used to culture the single CTCs with the small molecule compound is changed every three days with fresh compound.

7. The method of claim 1, wherein the CTC cell proliferation is evaluated every 6 days.

8. The method of claim 1, wherein the CTC cell proliferation is evaluated from 24 days.

9. A method to increase proliferation of circulating tumor cells (CTCs) comprising:
culturing CTCs in a medium comprising N-Acetyl-L-Cysteine (NAC), P1C2, and/or diclofenac sodium for about 14 to 30 days.

10. The method of claim 9, wherein the medium comprises about 250-350 μM of NAC.

11. A method to inhibit the proliferation and/or decrease the survivability of circulating tumor cells (CTCs) comprising:
contacting the CTCs with a therapeutically effective amount of a compound of Formula I: 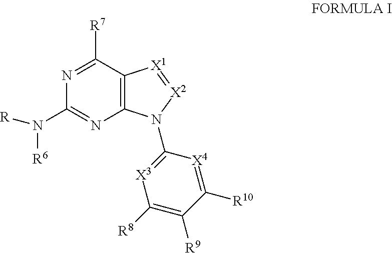
wherein R is an unsubstituted or substituted heterocycle, unsubstituted or substituted aryl, unsubstituted or substituted cycloalkyl, or unsubstituted or substituted cycloalkenyl, X¹ is N or CR¹¹, X² is N or CR¹², X³ is N or CR¹³, X⁴ is N or CR¹⁴; R⁶-R⁷ are each independently H, optionally substituted (C₁-C₆)alkyl, an optionally substituted (C₁-C₆) hetero-alkyl, an optionally substituted (C₁-C₆)alkenyl, an optionally substituted (C₁-C₆) hetero-alkenyl, an optionally substituted (C₁-C₆)alkynyl, an optionally substituted (C₁-C₆) hetero-alkynyl, an optionally substituted aryl, an optionally substituted (C₃-C₆)cycloalkyl, an optionally substituted (C₃-C₆)cycloalkenyl, and an optionally substituted heterocycle; R⁶-R¹⁰ are each independently H, optionally substituted (C₁-C₆)alkyl, —(CH₂)_{y}-nitrile; R¹¹-R¹⁴ are each independently H, optionally substituted (C₁-C₆)alkyl, an optionally substituted (C₁-C₆) hetero-alkyl, an optionally substituted (C₁-C₆)alkenyl, an optionally substituted (C₁-C₆) hetero-alkenyl, an optionally substituted (C₁-C₆)alkynyl, an optionally substituted (C₁-C₆) hetero-alkynyl, an optionally substituted aryl, an optionally substituted (C₃-C₆)cycloalkyl, an optionally substituted (C₃-C₆)cycloalkenyl, and an optionally substituted heterocycle; and y is an integer selected from 0, 1, 2, 3, 4, 5, and 6.

12. The method of claim 11, wherein the compound comprises a Formula I(a): 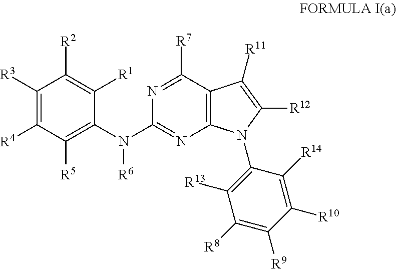
wherein R¹-R⁵ are each independently selected from H, a (C₁-C₆)alkoxy, optionally substituted (C₁-C₆)alkyl, an optionally substituted (C₁-C₆) hetero-alkyl, an optionally substituted (C₁-C₆)alkenyl, an optionally substituted (C₁-C₆) hetero-alkenyl, an optionally substituted (C₁-C₆)alkynyl, an optionally substituted (C₁-C₆) hetero-alkynyl, an optionally substituted aryl, an optionally substituted (C₃-C₆)cycloalkyl, an optionally substituted (C₃-C₆)cycloalkenyl, and an optionally substituted heterocycle; R⁶-R⁷ are each independently H, optionally substituted (C₁-C₆)alkyl, an optionally substituted (C₁-C₆) hetero-alkyl, an optionally substituted (C₁-C₆)alkenyl, an optionally substituted (C₁-C₆) hetero-alkenyl, an optionally substituted (C₁-C₆)alkynyl, an optionally substituted (C₁-C₆) hetero-alkynyl, an optionally substituted aryl, an optionally substituted (C₃-C₆)cycloalkyl, an optionally substituted (C₃-C₆)cycloalkenyl, and an optionally substituted heterocycle; R⁸-R¹⁰ are each independently H, optionally substituted (C₁-C₆)alkyl, —(CH₂)_{y}-nitrile; R¹¹-R¹⁴ are each independently H, optionally substituted (C₁-C₆)alkyl, an optionally substituted (C₁-C₆) hetero-alkyl, an optionally substituted (C₁-C₆)alkenyl, an optionally substituted (C₁-C₆) hetero-alkenyl, an optionally substituted (C₁-C₆)alkynyl, an optionally substituted (C₁-C₆) hetero-alkynyl, an optionally substituted aryl, an optionally substituted (C₃-C₆)cycloalkyl, an optionally substituted (C₃-C₆)cycloalkenyl, and an optionally substituted heterocycle; and y is an integer selected from 0, 1, 2, 3, 4, 5, and 6.

13. The method of claim 11, wherein the compound comprises Formula II:

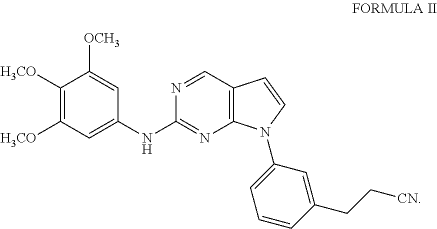 [SMILES: COc1cc(Nc2ncc3ccn(-c4cccc(CCC#N)c4)c3n2)cc(OC)c1OC]

14. The method of claim 11, wherein the method is carried out in vitro, ex vivo or in vivo.

15. The method of claim 11, wherein a pharmaceutical composition comprises the compound of Formula I, I(a) or II.

16. The method of claim 15, wherein the pharmaceutical composition comprising a therapeutically effective amount of the compound of Formula I, I(a) or II and is administered in vivo to a subject in need thereof.

17. The method of claim 16, wherein the subject has advanced metastatic cancer.

18. A method of screening for an effective anti-cancer therapy, the method comprising obtaining CTCs from a subject blood;
culturing the CTCs in about 300 μM of NAC in culture media for about 24 days to expand the CTCs;
culturing the expanded CTCs in media lacking NAC;
culturing the expanded CTCs in media comprising a test anti-cancer agent;
determining a criteria of the CTCs selected from the group consisting of cell growth, apoptosis, migration, infiltration, proliferation and gene expression wherein an inhibition of cell growth, migration, infiltration, or proliferation, or increase in apoptosis is indicative of an effective anti-cancer agent for treatment of the subject.

19. The method of claim 18, wherein the anti-cancer agent is selected from a chemotherapeutic agent, a small molecule agent, and an anti-cancer biological agent.
